(19) United States
(12) Patent Application Publication
(10) Pub. No.: US 2010/0249590 A1
(43) Pub. Date: Sep. 30, 2010

(54) ULTRASONIC DIAGNOSIS APPARATUS AND ULTRASONIC IMAGE GENERATING METHOD

(76) Inventors: Yuko KANAYAMA, Otawara-shi (JP); Naohisa KAMIYAMA, Otawara-shi (JP)

Correspondence Address: OBLON, SPIVAK, MCCLELLAND MAIER & NEUSTADT, L.L.P., 1940 DUKE STREET, ALEXANDRIA, VA, 22314, US

(21) Appl. No.: 12/749,918

(22) Filed: Mar. 30, 2010

(30) Foreign Application Priority Data
Mar. 31, 2009 (JP) 2009-085883

(51) Int. Cl. A61B 8/14

(52) U.S. Cl. 600/442

(57) ABSTRACT

The composite wave obtained by combining a fundamental wave having the center frequency f1 and a fundamental wave having the center frequency f2 is transmitted at least twice for each scanning line while performing phase modulation, and received an echo signal corresponding to each transmission for each scanning line. An echo signal in which harmonic components are canceled out, is extracted for each scanning line, by performing subtraction processing between echo signals which are obtained in this manner and respectively correspond to at least two transmissions. A signal strength difference (or a signal ratio if logarithmic compression is not performed) at each position on the scanned cross-section is calculated by using the f1 frequency component and f2 frequency component contained in the extracted echo signal. An attenuation image representing the signal strength difference at each position on the scanned cross-section is generated by using the calculated signal strength difference.

### CROSS-REFERENCE TO RELATED APPLICATIONS

This application is based upon and claims the benefit of priority from prior Japanese Patent Application No. 2009-085883, filed Mar. 31, 2009, the entire contents of which are incorporated herein by reference.

### BACKGROUND OF THE INVENTION

1. Field of the Invention

The present invention relates to an ultrasonic diagnosis apparatus which captures a tomogram of an organ by scanning the interior of a living body and diagnoses a disease and the like, and an ultrasonic image generating method.

2. Description of the Related Art

An ultrasonic diagnosis apparatus is a diagnosis apparatus which displays an image based on vital information. This apparatus is used as a useful apparatus for noninvasive real-time observation at low cost without exposure to radiation as compared with other types of image diagnosis apparatuses such as an X-ray CT apparatus. Ultrasonic diagnosis apparatuses are used in a wide range including diagnosis of circulatory organs such as the heart, abdominal regions such as the liver and kidney and peripheral vessels, diagnosis in obstetrics and gynecology, and breast cancer diagnosis.

An object as an ultrasonic diagnosis target is a living body. A biological tissue has a unique attenuation characteristic. Ultrasonic waves transmitted to an object propagate in the living body while being attenuated. If this attenuation amount is large, a sufficient echo signal cannot be received during the ultrasonic propagation. In general, the echo signal strength decreases due to attenuation toward deeper regions. For this reason, many recent ultrasonic diagnosis apparatuses are equipped with a function called STC (Sensitivity Time Control) which changes the gain in accordance with the depth. Recently, in addition, a function of automatically adjusting this STC has been popularized. This function performs analysis of reception signal strength (or in the lateral direction) for each depth and calculates a coefficient for each depth so as make reception signals constant.

Large tissue attenuation of an echo signal can be a factor that hinders the acquisition of diagnosis information. On the other hand, it is often the case that the characteristics of a biological tissue are observed by observing how an echo signal is attenuated. Take the liver for example. An object which excessively attenuates an echo signal has many lipid droplets contained in the liver and hence is inferred to be a fatty liver. The same result sometimes is obtained in the case of hepatocirrhosis.

The attenuation characteristics also depend on the frequency. For this reason, different transmission/reception frequencies lead to different behaviors of signal attenuation. Obviously, manually or automatically changing STC will change the luminance level at each depth. Under these circumstances, it is surmised that the ultrasonic image data finally acquired in ultrasonic image diagnosis has been influenced by tissue attenuation and signal processing by STC. For this reason, it is generally difficult to quantitatively evaluate the attenuation of an echo signal by using only a luminance change using the display image obtained by an ultrasonic diagnosis apparatus. A recent ultrasonic diagnosis apparatus has exhibited a dramatic improvement in signal reception sensitivity, and can obtain sufficient depths of field in most objects. The operator cannot therefore actively perceive the degrees of attenuation.

Several techniques have been proposed to solve these problems. For example, Jpn. Pat. Appln. KOKAI Publication No. 3-24868 discloses a technique of divisionally transmitting and receiving ultrasonic pulses in different frequency bands twice in the same direction. Based on the fact that the tissue attenuation amount varies depending on the frequency, this technique infers the attenuation constant of a medium by comparing the attenuation amounts of two pulses. Jpn. Pat. Appln. KOKAI Publication No. 3-24868 also discloses a technique of adding attenuation information to a conventional tomogram by extracting two different frequency band components contained in a reception signal upon performing transmission/reception once in the same direction, and weighting and adding the respective signals. This technique can be easily implemented by one transmission/reception cycle.

As described above, the conventional technique uses the phenomenon that the attenuation amount due to ultrasonic propagation increases with an increase in frequency. The conventional technique, however, gives no consideration to the phenomenon that harmonics occur due to ultrasonic propagation, and hence cannot accurately and easily acquire the attenuation amount of ultrasonic waves as effective diagnosis information.

That is, an ultrasonic pulse transmitted into a living body exhibits waveform distortion in the process of propagation, and the harmonic components of the pulse waveform are gradually amplified. The fact that considerable amplification of harmonic components occurs in the living body indicates that it is difficult to simply compare two frequency components. This is because while a given frequency component is attenuated by tissue attenuation in the process of ultrasonic propagation in the living body, the component is amplified by tissue harmonics produced by a frequency component ½ the given frequency component. In spite of all efforts, therefore, it is difficult to estimate an accurate attenuation amount by comparing two different frequency components.

Recently, there has been developed an imaging technique paying attention to the phenomenon that harmonics are generated in the process of ultrasonic propagation in a living body. This technique is called tissue harmonic imaging (THI), which acquires an ultrasonic image with high contrast resolution and high spatial resolution by extracting and imaging only harmonic components due to ultrasonic propagation.

### BRIEF SUMMARY OF THE INVENTION

The present invention is contrived in consideration of the above-described circumstances, and an object of the invention is to provide an ultrasonic diagnostic apparatus and an ultrasonic image generating method capable of removing the influence of harmonics from an attenuation image and more accurately evaluating an attenuation amount of a propagating ultrasonic wave in an object.

According to an aspect of the present invention, there is provided an ultrasonic diagnostic apparatus comprising: a transmission unit which transmits a composite ultrasonic wave obtained by combining at least a first ultrasonic wave having a first center frequency with a second ultrasonic wave having a second center frequency different from the first center frequency at least twice in each of a plurality of directions in an object while modulating a phase; an ultrasonic reception unit which receives, from the object, an echo signal corresponding to each of the at least two transmissions in each of the plurality of directions; a signal extraction unit which extracts a first echo signal corresponding to the first ultrasonic wave and a second echo signal corresponding to the second ultrasonic wave after canceling out harmonics by performing subtraction processing between the echo signals respectively corresponding to the at least two transmissions in each of the plurality of directions; and an image generating unit which generates an attenuation image representing attenuation of an ultrasonic wave propagating in the object by using the first echo signal and the second echo signal.

According to another aspect of the present invention, there is provided an ultrasonic image generating method which is executed by using an ultrasonic diagnosis apparatus, the method comprising: transmitting a composite ultrasonic wave obtained by combining at least a first ultrasonic wave having a first center frequency with a second ultrasonic wave having a second center frequency different from the first center frequency in each of a plurality of directions at least twice in an object while modulating a phase; receiving, from the object, an echo signal corresponding to each of the at least two transmissions in each of the plurality of directions; extracting a first echo signal corresponding to the first ultrasonic wave and a second echo signal corresponding to the second ultrasonic wave after canceling out harmonics by performing subtraction processing between the echo signals respectively corresponding to the at least two transmissions in each of the plurality of directions; and generating an attenuation image representing attenuation of an ultrasonic wave propagating in the object by using the first echo signal and the second echo signal.

### BRIEF DESCRIPTION OF THE SEVERAL VIEWS OF THE DRAWING

FIG. 1 is a block diagram showing the block arrangement of an ultrasonic diagnosis apparatus according to the first embodiment;

FIG. 2 is a flowchart showing a procedure for attenuation image generation processing according to the first embodiment;

FIGS. 3A and 3B are views for explaining conventional ultrasonic transmission/reception;

FIGS. 4A and 4B are views for explaining a composite wave used in Step S2 and made up of a first fundamental wave and a second fundamental wave;

FIG. 5 is a graph for explaining the effect of the subtraction performed in step S3, on the basis of a specific example of actual measurement;

FIG. 6 is a conceptual graph showing how the frequency spectrum of an echo signal at a given depth position differs between the case where the subtraction in Step S3 is performed and the case where the subtraction is not performed;

FIG. 7 is a view showing an example of the display form of an attenuation image;

FIG. 8 is a view showing an example of the parallel display of one of first, second, and third ultrasonic images 71 and an attenuation image 72;

FIG. 9 is a view showing an example of a color scale bar used when an attenuation image is display in color;

FIG. 10 is a flowchart showing a procedure for attenuation image generation processing according to the second embodiment; and

FIGS. 11A and 11B are graphs each showing an example of f1 frequency components and f2 frequency components before and after f1 attenuation correction in step S14.

### DETAILED DESCRIPTION OF THE INVENTION

The first and second embodiments of the present invention will be described below with reference to the views of the accompanying drawing. Note that the same reference numerals denote constituent elements having almost the same functions and arrangements, and a repetitive description will be made only when required.

### First Embodiment

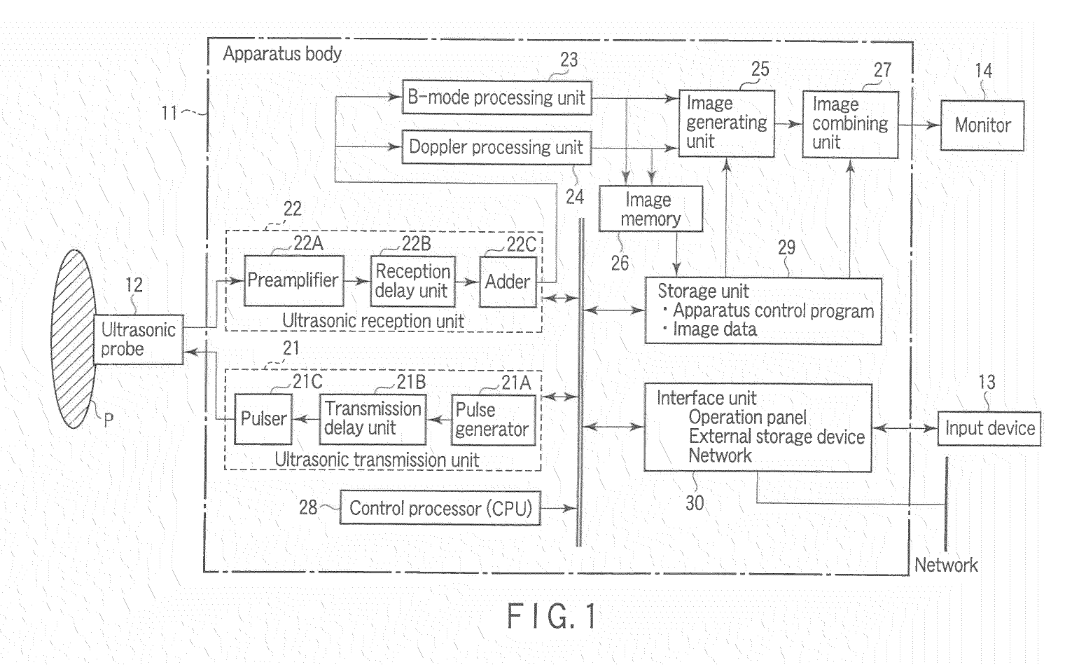

FIG. 1 is a block arrangement of an ultrasonic diagnosis apparatus according to this embodiment. As shown in FIG. 1, an ultrasonic diagnosis apparatus body 11 includes an ultrasonic probe 12, an input device 13, a monitor 14, an ultrasonic transmission unit 21, an ultrasonic reception unit 22, a B-mode processing unit 23, a Doppler processing unit 24, an image generating unit 25, an image memory 26, an image combining unit 27, a control processor 28, a storage unit 29, and other interface units 30. The ultrasonic transmission unit 21, ultrasonic reception unit 22, and the like incorporated in the apparatus body 11 are sometimes implemented by hardware such as integrated circuits and other times by software programs in the form of software modules. The function of each constituent element will be described below.

The ultrasonic probe 12 includes a plurality of piezoelectric vibrators which generate ultrasonic waves based on driving signals from the ultrasonic transmission unit 21 and convert reflected waves from an object into electrical signals, a matching layer provided for the piezoelectric vibrators, and a backing member which prevents ultrasonic waves from propagating backward from the piezoelectric vibrators. When ultrasonic waves are transmitted from the ultrasonic probe 12 to an object P, the transmitted ultrasonic waves are sequentially reflected by the discontinuity surface of acoustic impedance of an internal body tissue, and are received as an echo signal by the ultrasonic probe 12. The amplitude of this echo signal depends on an acoustic impedance difference on the discontinuity surface by which the echo signal is reflected. The echo produced when a transmitted ultrasonic pulse is reflected by the surface of a moving blood flow, cardiac wall, or the like is subjected to a frequency shift depending on the velocity component of the moving body in the ultrasonic transmission direction due to the Doppler effect.

The input device 13 is connected to the apparatus body 11 and includes various types of switches, buttons, a trackball, a mouse, and a keyboard which are used to input, to the apparatus body 11, various types of instructions and conditions, an instruction to set a region of interest (ROI), various types of image quality condition setting instructions, and the like from an operator.

The monitor 14 displays an image indicating morphological information in the living body, an image indicating blood flow information, an attenuation image (to be described below), and the like based on video signals from the image generating unit 25.

The ultrasonic transmission unit 21 includes a pulse generator 21A, a transmission delay unit 21B, and a pulser 21C. The pulse generator 21A repetitively generates rate pulses for the formation of transmission ultrasonic waves at a predetermined rate frequency fr Hz (period: 1/fr sec). The transmission delay unit 21B gives each rate pulse a delay time necessary to focus an ultrasonic wave into a beam and determine transmission directivity for each channel. The pulser 21C applies a driving pulse to the ultrasonic probe 12 at the timing based on this rate pulse.

The ultrasonic reception unit 22 includes a preamplifier 22A, an A/D converter (not shown), a reception delay unit 22B, and an adder 22C. The preamplifier 22A amplifies an echo signal captured via the probe 12 for each channel. The reception delay unit 22B gives the amplified echo signals delay times necessary to determine reception directivities. The adder 22C then performs addition processing for the signals. With this addition, the reflection component of the echo signal from the direction corresponding to the reception directivity is enhanced, and a synthetic beam for ultrasonic transmission/reception is formed in accordance with the reception directivity and transmission directivity. The ultrasonic reception unit 22 executes subtraction processing (to be described later) to extract fundamental wave components from which the influences of harmonics are removed.

The B-mode processing unit 23 receives the echo signal from the reception unit 22, and performs logarithmic amplification, envelope detection processing, and the like, thereby generating data whose signal strength is represented by a luminance level. Although not shown, the B-mode processing unit 23 includes a line memory which temporarily stores echo signals, and hence can perform addition processing, subtraction processing, and the like for arbitrary two echo signals. An output from the B-mode processing unit 23 is transmitted to the image generating unit 25. The monitor 14 then displays the output as a B-mode image representing the strength of a reflected wave as a luminance. The B-mode processing unit 23 also generates attenuation image data by executing difference processing or the like (to be described later).

The Doppler processing unit 24 frequency-analyzes velocity information from the echo signal received from the reception unit 22 to extract a blood flow, tissue, and contrast medium echo component by the Doppler effect, and obtains blood flow information such as an average velocity, variance, and power at multiple points. The obtained blood flow information is sent to the image generating unit 25. The monitor 14 then displays the information as an average velocity image, a variance image, a power image, or a composite image of them in color.

The image generating unit 25 generates an ultrasonic diagnosis image as a display image by converting the scanning line signal string obtained by ultrasonic scanning into a scanning line signal string in a general video format typified by a TV format. The image generating unit 25 is equipped with a memory which stores image data. For example, after diagnosis, the operator can read out an image recorded during examination. Note that data before it is input to the image generating unit 25 is sometimes called “raw data”.

The image memory 26 includes a memory which stores image data received from the image generating unit 25. The operator can read out this image data after, for example, diagnosis. Such an image can be played back as a still image or as a moving image using a plurality of frames. The image memory 26 also stores an output signal (called an RF (Radio Frequency) signal) immediately after the ultrasonic reception unit 22, an image luminance signal after passage through the reception unit 22, other kinds of raw data, image data acquired via a network, and the like, as needed.

The control processor 28 is a control unit which has a function as an information processing apparatus (computer) and controls the operation of this ultrasonic diagnosis apparatus body. The control processor 28 reads out control programs for the execution of an attenuation image generating function (to be described later) and the like from the storage unit 29, and executes computation/control and the like associated with various kinds of processing.

The storage unit 29 is used to archive control programs for executing a scan sequence (to be described later), image generation processing, and display processing, diagnosis information (a patient ID, findings by a doctor, and the like), a diagnosis protocol, transmission/reception conditions, a program for implementing an attenuation image generating function (to be described later), and other data. This storage unit is also used to, for example, archive images in the image memory 26. Data in the storage unit 29 can be transferred to an external peripheral apparatus via the interface unit 31.

The interface unit 30 is an interface associated with the input unit 13, a network, and a new external storage device (not shown). The interface unit 30 can transfer data such as ultrasonic images, analysis results, and the like obtained by this apparatus to another apparatus via a network.

### (Attenuation Image Generating Function)

The attenuation image generating function which the ultrasonic diagnosis apparatus body 11 according to this embodiment has will be described next. This function uses a composite wave obtained by combining at least two fundamental waves having different center frequencies. The function transmits this composite wave at least twice while performing phase modulation for each scanning line and receives an echo signal corresponding to each transmission for each scanning line. The function extracts an echo signal in which harmonic components are canceled out by performing subtraction processing, for each scanning line, between echo signals corresponding to at least two transmissions, respectively, and obtained in the above manner. The function then generates an image (attenuation image) representing the attenuation of ultrasonic waves propagating in the object by using the extracted echo signal.

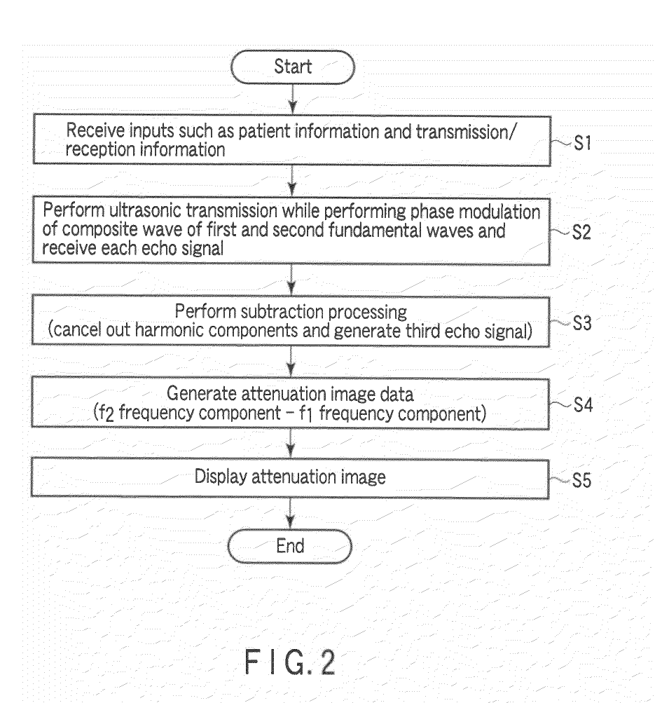

FIG. 2 is a flowchart showing a procedure for processing (attenuation image generation processing) complying with the attenuation image generating function according to this embodiment. The contents of processing in each step will be described below.

### [Reception of Inputs Including Patient Information and Transmission/Reception Conditions: Step S1]

First of all, the control processor 28 receives inputs including patient information, and transmission conditions and reception conditions (transmission/reception conditions) via the input device 13. In this case, the control processor 28 receives transmission conditions for the transmission of an ultrasonic pulse containing a plurality of frequency components in a relatively high frequency band, the number of times of ultrasonic transmission/reception to be performed a plurality of number of times accompanied by phase modulation executed for each scanning line, and the like. In this embodiment, for the sake of concreteness, assume that ultrasonic transmission/reception accompanied by phase modulation is executed twice for each scanning line. However, the present invention is not limited to this example. The operator can input an arbitrary number of times.

### [Ultrasonic Transmission/Reception Accompanied by Phase Modulation: Step S2]

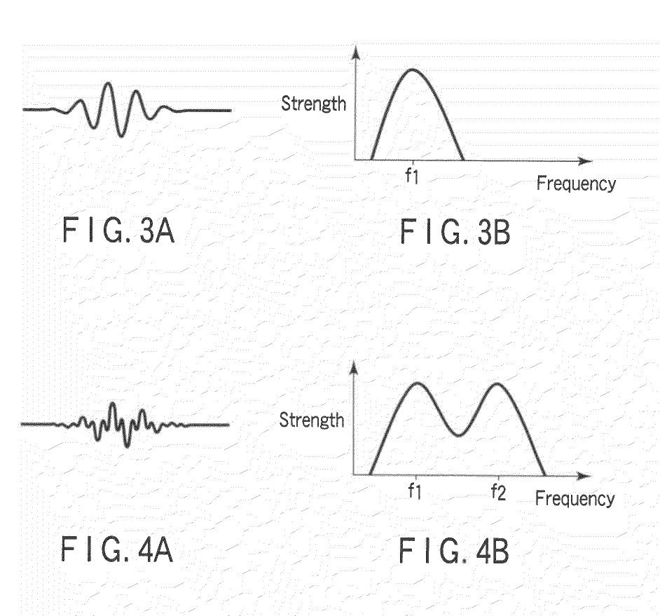

The control processor 28 controls the ultrasonic transmission unit 21 to execute ultrasonic transmission in accordance with the transmission conditions input in step S1. The ultrasonic pulse transmitted from the ultrasonic transmission unit 21 is a pulse containing a plurality of frequency components in a relatively wide frequency band for the proper analysis of an attenuation amount (to be described later). For example, the ultrasonic pulse waveform to be transmitted in step S2 is a composite ultrasonic pulse obtained by linearly adding (combining) fundamental waves having two different center frequencies f1 and f2 (f1<f2) as shown in FIG. 4A, unlike a conventional ultrasonic pulse having a waveform based on a single frequency as shown in FIG. 3A. The frequency band of an ultrasonic pulse to be transmitted with this waveform has a spectrum like that shown in FIG. 4B which has a wide frequency band. In contrast, an ultrasonic pulse which is conventionally used has a spectrum in a band narrower than that used in step S2, as shown in FIG. 3B. The ultrasonic transmission unit 21 performs ultrasonic transmission twice for each scanning line while performing phase modulation (i.e., 180° phase modulation) of the above composite ultrasonic pulse so as to reverse the polarity of the waveform in the first transmission relative to that in the second transmission (make the waveforms in the first and second transmissions have opposite polarities).

This embodiment has exemplified the case in which the apparatus uses the composite ultrasonic pulse obtained by linearly adding (combining) fundamental waves having the two different center frequencies f1 and f2. However, the composite ultrasonic pulse to be used is not limited to this. A composite ultrasonic pulse may be generated by linearly adding fundamental waves having three or more different center frequencies.

[Subtraction Processing (Extraction of f1 Frequency Component and f2 Frequency Component): Step S3]

The ultrasonic reception unit 22 receives an echo signal (first echo signal) corresponding to the first transmitted ultrasonic wave and an echo signal (second echo signal) corresponding to the second transmitted ultrasonic wave at predetermined timings, respectively, and subtracts one from the other (for example, subtracts the second echo signal from the first echo signal). This subtraction processing cancels out harmonics and generates an echo signal (third echo signal) in which the f1 frequency component and the f2 frequency component are enhanced and extracted by subtraction.

This embodiment subtracts echo signals based on two pulses having waveforms with opposite polarities from each other. The purpose of this operation is to remove the influence of the occurrence of harmonic components due to waveform distortion during ultrasonic propagation. That is, for example, in conventional ultrasonic transmission/reception like that shown in FIG. 3A, a harmonic component of the fundamental wave having the center frequency f1 occurs in the f2 frequency band during ultrasonic propagation in the living body. Since nonlinear distortions are accumulated in the process of ultrasonic propagation, the amount of distortions accumulated increases with an increase in distance. For this reason, the f2 frequency component is reduced by tissue attenuation on one hand, and is increased due to the accumulation of harmonic components on the other hand. This makes it impossible to reflect an accurate attenuation amount.

The third echo signal obtained by subtraction in step S2 is a signal in which the second harmonic component due to ultrasonic propagation in the living body is canceled out, and only a fundamental wave component remains. This makes it possible to effectively extract effects due to tissue attenuation.

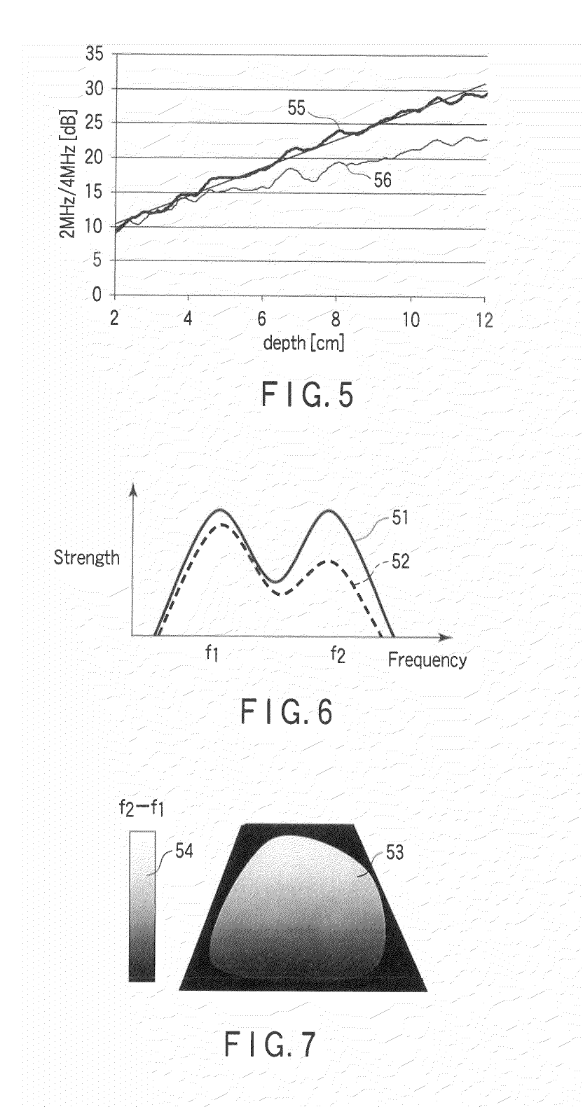

FIG. 5 shows a specific example of actual measurement associated with subtraction in step S2. Referring to FIG. 5, a 2 MHz band component and a 4 MHz band component are extracted from an echo signal from a phantom having an attenuation of 0.5 dB/cm/MHz, and the ratio between them is computed for each observation depth. A computation result 55 corresponding to the third echo signal indicates that an almost uniform (linear) increase is observed at each depth, and the attenuation amount is almost equal to the value calculated from the nominal value of the phantom. In contrast, a result 56 corresponding to a conventional echo signal indicates that the signal ratio increases with an increase in depth, and the degree of the increase is smaller than that of the result 55 from the third echo signal. Obviously, this result indicates the influence of the occurrence of harmonics.

Adding echo signals based on two pulses whose waveforms have opposite polarities will cancel the fundamental wave signals contained in transmitted ultrasonic waves and extract only nonlinear components generated by distortions during ultrasonic propagation in the living body. This technique is widely used in the tissue harmonic imaging (THI) method.

### [Generation of Attenuation Image Data: Step S4]

The B-mode processing unit 23 extracts an f1 frequency component and an f2 frequency component from the third echo signal by using two or more types of bandpass filters. The B-mode processing unit 23 calculates the signal strength difference (or a signal strength ratio if logarithmic compression is not performed) between the extracted f1 frequency component and f2 frequency component at each position on a scanned cross-section. In addition, the B-mode processing unit 23 generates image data (attenuation image data) representing the signal strength difference at each position on the scanned cross-section by using the calculated signal strength differences.

Note that this embodiment has exemplified the case in which ultrasonic transmission is performed by using the composite ultrasonic pulse obtained by combining the f1 frequency component and the f2 frequency component. Assume that a composite ultrasonic pulse composed of three or more types of frequency components is transmitted, and the third echo signal obtained from each echo signal received as a result of the transmission is used. In this case, it is possible to calculate the above signal ratio by using, for example, the average value of signal ratios obtained by different combinations of frequency components. In addition, a signal difference may be obtained by division of RF signals before logarithmic transformation or difference computation after logarithmic transformation.

### [Display of Attenuation Image: Step S5]

The image generating unit 25 generates an attenuation image by using the attenuation image data acquired from the B-mode processing unit 23. The image combining unit 27 combines the generated attenuation image with predetermined information. The monitor 14 displays the resultant image in a predetermined form.

The characteristics of an attenuation image will be described below. First of all, a basic principle is that an echo signal propagating in a living body is attenuated with an increase in frequency. According to this principle, if, for example, the frequency spectrum of an echo signal at a given position in a near distance region (i.e., a region near the surface of the object) in FIG. 6 is denoted by reference numeral 51, the frequency spectrum of an echo signal at a given position in a far distance region (i.e., a deep region from the surface of the object) can be conceptually expressed like a spectrum 52. That is, since the attenuation amount in a high frequency band f2 is larger than that in a low frequency band f1, if the signal strength difference between the f1 band the f2 band is computed, it is expected that the difference is small in the near distance region and is large in the far distance region. Therefore, an attenuation image generated by using attenuation image data as the difference between the f1 frequency component and the f2 frequency component can visualize the attenuation state of the ultrasonic wave in accordance with the depth in, for example, a form that the image becomes darker with an increase in depth. Obviously, an attenuation image depends on the magnitude of tissue attenuation. In the case of a medium with small attenuation, e.g., water, the difference between f1 and f2 is almost constant regardless of depth.

FIG. 7 is a view showing an example of the display form of an attenuation image. In the example shown in FIG. 7, an attenuation image 53 is displayed together with a scale bar 54 indicating the degree of difference (in this case, the color of the bar becomes darker with an increase in the attenuation amount of an ultrasonic wave with the frequency f2).

The image generating unit 25 can generate the first ultrasonic image corresponding to a fundamental wave having a center frequency f1 and the second ultrasonic image corresponding to a fundamental wave having a center frequency f2 by using the first and second echo signals processed by the B-mode processing unit 23. The image generating unit 25 can also generate the third ultrasonic image containing both an f1 frequency component and an f2 frequency component by using the sum echo signal obtained by adding the first and second echo signals processed by the B-mode processing unit 23. It is possible to display these ultrasonic images singly or together with an attenuation image in a predetermined form.

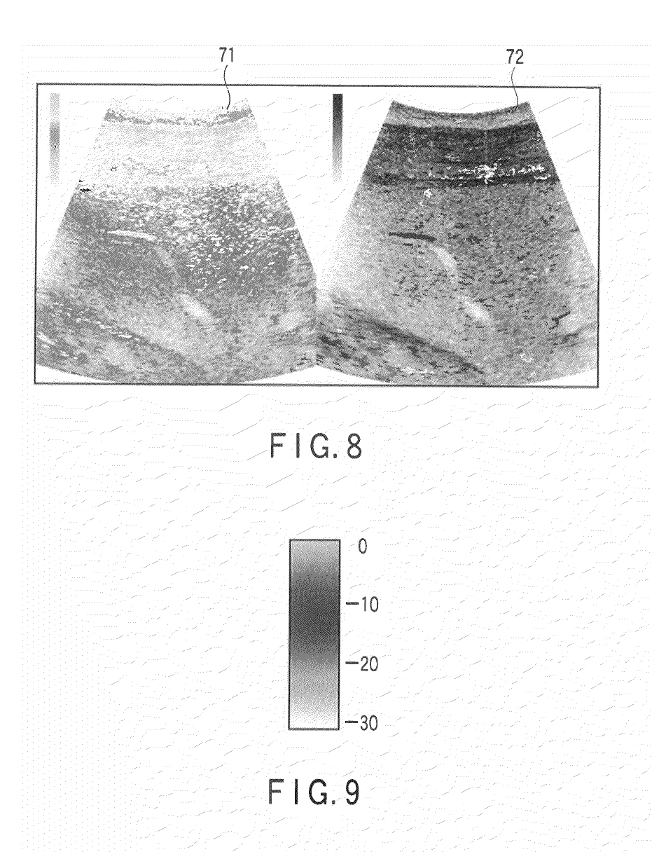

FIG. 8 is a view showing an example of the parallel display of one of first, second, or third ultrasonic images 71 and an attenuation image 72. In general, a B-mode tomogram having undergone attenuation correction is more suitable for the observation of a tissue structure. The display form shown in FIG. 8 allows to easily and quickly observe the tissue structure on the normal ultrasonic image 71 and the attenuation state of the ultrasonic wave corresponding to the depth on the attenuation image 72.

Note that since it is desirable to express the state of attenuation of an attenuation image in a more enhanced manner, it is possible to perform color display depending on the attenuation amount (i.e., display with colors assigned in accordance with the degrees of attenuation). In this case, it is preferable to simultaneously display a color scale bar indicating the correspondence relationship between attenuation amounts and respective colors as shown in FIG. 9. This color scale bar can also display numerical values as computation results to indicate more quantitative information. In addition, the present invention is not limited to this, and may superimpose and display one of the first, second, and third ultrasonic images and an attenuation image.

### (Effects)

According to the above arrangement, the following effects can be obtained.

This ultrasonic diagnosis apparatus transmits the composite wave obtained by combining a fundamental wave having the center frequency f1 and a fundamental wave having the center frequency f2 at least twice for each scanning line while performing phase modulation, and receives an echo signal corresponding to each transmission for each scanning line. This apparatus extracts an echo signal in which harmonic components are canceled out, for each scanning line, by performing subtraction processing between echo signals which are obtained in this manner and respectively correspond to at least two transmissions. The apparatus then calculates a signal strength difference (or a signal strength ratio if logarithmic compression is not preformed) at each position on the scanned cross-section by using the f1 frequency component and f2 frequency component contained in the extracted echo signal. In addition, the apparatus uses the calculated signal strength difference to generate an attenuation image representing the signal strength difference at each position on the scanned cross-section by using a brightness. This makes it possible to generate an attenuation image from which the influence of harmonics is removed and to easily evaluate the degree of fatty change in the liver or the ratio between the mammary fatty tissue and the mammary glands.

In addition, it is possible to display a normal ultrasonic image and an attenuation image in a desired form such as parallel display or superimposed display. In image observation, therefore, it is possible to easily and quickly observe a tissue structure on a normal ultrasonic image and the attenuation state of an ultrasonic wave corresponding to each depth on an attenuation image. It is also possible to perform color display corresponding to the degree of attenuation and hence to provide an attenuation image with high visibility.

### Second Embodiment

The second embodiment of the present invention will be described next. An ultrasonic diagnosis apparatus according to this embodiment uses the echo signals acquired by the same ultrasonic transmission/reception as that in the first embodiment, and extracts an echo signal in which harmonic components are canceled out by performing subtraction processing between the echo signals for each scanning line. This apparatus generates an attenuation image by, for example, executing the same attenuation correction as that executed for an ultrasonic image using an f1 frequency component for an ultrasonic image using an f2 frequency component (or executing the same attenuation correction as that executed for an ultrasonic image using an f2 frequency component for an ultrasonic image using an f1 frequency component) by using the extracted echo signal. For the sake of concreteness, the following is a case in which f1 attenuation correction executed for an ultrasonic image using an f1 frequency component is also executed for an ultrasonic image using an f2 frequency component.

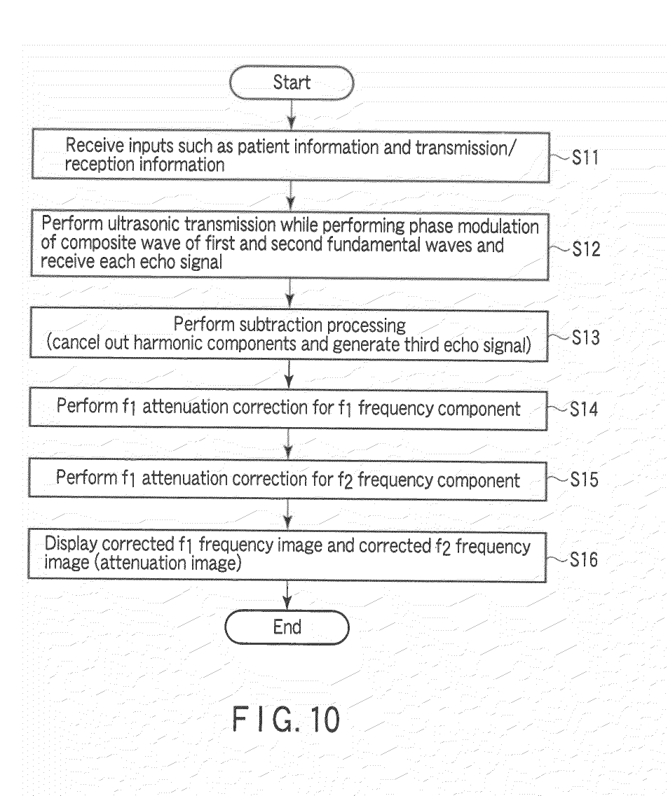

FIG. 10 is a flowchart showing a sequence for attenuation image generation processing according to the second embodiment. The contents of processing in each step will be described below. Since each process in steps S11 to S13 in FIG. 10 is almost the same as that in steps S1 to S3 shown in FIG. 2, the contents of each process in steps S14 to S16 will be described below.

[f1 Attenuation Correction for f1 Frequency Component: Step S14]

A B-mode processing unit 23 then extracts the f1 frequency component and the f2 frequency component from the third echo signal by using two or more types of bandpass filters. The B-mode processing unit 23 corrects the attenuation of the f1 frequency component inside the object with respect to the extracted f1 frequency component, and acquires a function (attenuation correction function) g(x, y) for making the f1 frequency component strength constant. The B-mode processing unit 23 then executes processing (f1 attenuation correction) for making the f1 frequency component strength constant by using the attenuation correction function g(x, y). The B-mode processing unit 23 uses the f1 frequency component after correction to generate image data (corrected f1 frequency component image data) representing the signal strength difference at each position on the scanned cross-section by using a brightness. The B-mode processing unit 23 further executes f1 attenuation correction by using the attenuation correction function g(x, y) for the extracted f2 frequency component, and uses the f2 frequency component after correction to generate image data (corrected f2 frequency component image data) representing the signal strength at each position on the scanned cross-section by using a brightness.

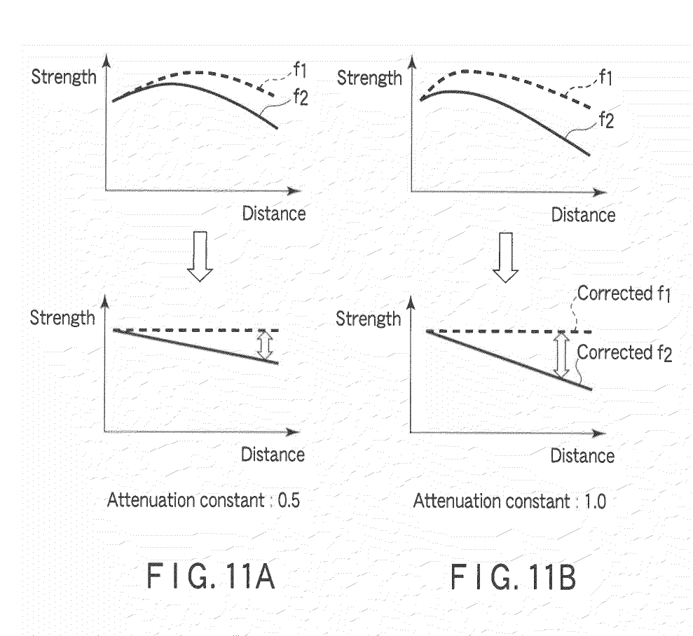

The characteristics of a corrected f2 frequency component image will be described below. In general, since an echo signal propagating in a living body is more attenuated with an increase in frequency, the f2 frequency component as a high-frequency component is more attenuated than the f1 frequency component inside the object, as shown in FIG. 6. Therefore, the difference between the f2 frequency component and the f1 frequency component increases with an increase in depth (distance) as indicated by the upper graph of FIG. 11A. In addition, when f1 attenuation correction for making the f1 frequency component strength constant is performed for the f2 frequency component, the corrected f2 frequency component is attenuated by an attenuation constant unique to the material of the object (0.5 in the case shown in FIG. 11A) relative to the f1 frequency component indicated by the dotted line with constant strength. Having such characteristics, the corrected f2 frequency component image corrected by using f1 attenuation correction which is an image where its brightness represents the signal strength difference at each position on the scanned cross-section, i.e., an attenuation image itself.

The object has an attenuation constant unique to the material it is made of. The subject method can measure or quantitatively analyze the attenuation constant unique to the object.

### [Display of Attenuation Image: Step S15]

An image generating unit 25 generates an attenuation image by using the attenuation image data acquired from the B-mode processing unit 23. An image combining unit 27 combines the generated attenuation image with predetermined information. A monitor 14 displays the resultant image in a predetermined form. The display forms to be used are the same as those in the first embodiment.

According to the above arrangement, this apparatus transmits the composite wave obtained by combining two ultrasonic waves respectively having center frequencies f1 and f2, twice for each scanning line, while performing phase modulation, and receives an echo signal corresponding to each transmission for each scanning line. The apparatus extracts an echo signal in which harmonic components are canceled out, for each scanning line, by performing subtraction processing between echo signals which are obtained in this manner and correspond to at least two transmissions. For example, f1 attenuation correction executed for an ultrasonic image using the f1 frequency component is also executed for an ultrasonic image using the f2 frequency component, thereby generating an attenuation image. This makes it possible to generate an attenuation image from which the influence of harmonics is removed and to easily evaluate the degree of fatty change in the liver and the ratio between the mammary fatty tissue and the mammary glands.

Note that the present invention is not limited to the above embodiments, and constituent elements can be variously modified and embodied at the execution stage within the spirit and scope of the invention. For example, each function associated with each embodiment can also be implemented by installing programs for executing the corresponding processing in a computer such as a workstation and mapping them in a memory. In this case, the programs which can cause the computer to execute the corresponding techniques can be distributed by being stored in recording media such as magnetic disks (Floppy® disks, hard disks, and the like), optical disks (CD-ROMs, DVDs, and the like), and semiconductor memories.

In addition, various inventions can be formed by proper combinations of a plurality of constituent elements disclosed in the above embodiments. For example, several constituent elements may be omitted from all the constituent elements disclosed in the above embodiments. Furthermore, constituent elements in the different embodiments may be properly combined.

What is claimed is:

1. An ultrasonic diagnosis apparatus comprising:
a transmission unit which transmits a composite ultrasonic wave obtained by combining at least a first ultrasonic wave having a first center frequency with a second ultrasonic wave having a second center frequency different from the first center frequency at least twice in each of a plurality of directions in an object while modulating a phase;
an ultrasonic reception unit which receives, from the object, an echo signal corresponding to each of the at least two transmissions in each of the plurality of directions;
a signal extraction unit which extracts a first echo signal corresponding to the first ultrasonic wave and a second echo signal corresponding to the second ultrasonic wave after canceling out harmonics by performing subtraction processing between the echo signals respectively corresponding to the at least two transmissions in each of the plurality of directions; and
an image generating unit which generates an attenuation image representing attenuation of an ultrasonic wave propagating in the object by using the first echo signal and the second echo signal.

2. The apparatus according to claim 1, wherein the image generating unit generates a third echo signal by subtraction processing using the first echo signal and the second echo signal, and generates the attenuation image by using the third echo signal.

3. The apparatus according to claim 1, wherein the image generating unit executes first correction processing for correcting attenuation of the first echo signal in the object, and generates the attenuation image by performing the first correction processing for the second echo signal.

4. The apparatus according to claim 1, further comprising a display unit which displays, in a predetermined form, an ultrasonic image representing a tissue structure of the object, which is generated by using one of the first echo signal and the second echo signal, and the attenuation image.

5. The apparatus according to claim 4, wherein the display unit performs color display of the attenuation image by colors assigning in accordance with degrees of attenuation.

6. An ultrasonic image generating method which is executed by using an ultrasonic diagnosis apparatus, the method comprising:
transmitting a composite ultrasonic wave obtained by combining at least a first ultrasonic wave having a first center frequency with a second ultrasonic wave having a second center frequency different from the first center frequency in each of a plurality of directions at least twice in an object while modulating a phase;
receiving, from the object, an echo signal corresponding to each of the at least two transmissions in each of the plurality of directions;
extracting a first echo signal corresponding to the first ultrasonic wave and a second echo signal corresponding to the second ultrasonic wave after canceling out harmonics by performing subtraction processing between the echo signals respectively corresponding to the at least two transmissions in each of the plurality of directions; and
generating an attenuation image representing attenuation of an ultrasonic wave propagating in the object by using the first echo signal and the second echo signal.

7. The method according to claim 6, wherein in the image generation, a third echo signal is generated by subtraction processing using the first echo signal and the second echo signal, and the attenuation image is generated by using the third echo signal.

8. The method according to claim 6, wherein in the image generation, first correction processing is executed for correcting attenuation of the first echo signal in the object, and the attenuation image is generated by performing the first correction processing for the second echo signal.

9. The method according to claim 6, further comprising displaying, in a predetermined form, an ultrasonic image representing a tissue structure of the object, which is generated by using one of the first echo signal and the second echo signal, and the attenuation image.

10. The method according to claim 9, wherein in the display, color display of the attenuation image is performed by assigning colors in accordance with degrees of attenuation.
